(19) United States
(12) Patent Application Publication
(10) Pub. No.: US 2004/0229296 A1
(43) Pub. Date: Nov. 18, 2004

(54) Etiology of cancer

(76) Inventors: Maher Nagib Nashed, Newark, DE (US); Suzy Fawzy Nashed, Newark, DE (US)

Correspondence Address: Maher N Nashed, MD, 32 Winding Rd, Newark, DE, 19702, US

(21) Appl. No.: 10/440,296

(22) Filed: May 16, 2003

(51) Int. Cl. G01N 33/574; A61K 31/66

(52) U.S. Cl. 435/7.23

(57) ABSTRACT

Cancer has been around for many decades. No one was able to find the cause. We do claim the cause of cancer and the treatment based on the scientific data presented.

### CROSS-REFERENCE TO RELATED APPLICATIONS

Not Applicable.

### THE RESEARCH IS NOT UNDER FEDERALLY SPONSORED RESEARCH AND DEVELOPMENT

### BACKGROUND AND DISCRIPTION

The Generalized Conditions of Decrease Cell Energy Can Include

A. Phosphate depletion (83).

B. Increased intracellular Calcium (114) with Calcium influx in the cell (55, 56, 83, 92).

C. Drugs or Chemicals. (mitochondrial inhibitors) cyanide, antimycin, oligomycin, CCCP, Amobarbital, Iodoacetate. (67,70)

D. Catabolic conditions and extensive burns (84).

And Localized Conditions Can Include

1. Hypoxia and local ischemia (52,65).

2. Mechanical Stimulation or inflammation (180, 50, 77).

The Effects of this Decrease in the Cell Energy will Include

1. Decrease in Ca pump which will lead to decrease Ca extrusion which will lead to increase in intracellular Ca (83)

2. Decrease in Na—K pump (83,97) which will lead to loss of k (120) and increase entry of Na with possible cotransport of sugars and amino acids and exchange with Mg i(102).

3. Activation of K ATP channels which will lead to K loss (1, 119, 120, 146, 187)

4. Diminish gap junction permeability (1,2) which is common in cancer (1, 3, 4, 5, 6, 7, 8, 9, 10, 11, 12, 13, 14, 15, 16, 17, 18, 19, 27, 28, 30, 32, 34, 39, 42)

5. Magnesium depletion caused by phosphate depletion. (81, 85, 87).

6. Potassium depletion caused by Mg depletion. (87,96) and phosphate depletion with decrease ATP (83).

### These Effects will Lead to Changes in a Cell to a Cancerous One as will be Explained Later

### A. Causes of Phosphate Depletion

1. Absorption of phosphate can be blocked by commonly used over-the-counter aluminum-, calcium-, and magnesium-containing antacids.

2. Diseases causing severe diarrhea or intestinal malabsorption.

3. Poor nutrition.

4. hyperparathyroidism,

5. Several genetic and acquired syndromes of phosphate wasting and associated skeletal abnormalities have been described.

6. Refeeding, short-term increases in cellular demand, such as in hungry bones syndrome, and acute respiratory alkalosis.

7. hospitalized patients, hypophosphatemia is observed in 1-5% of individuals and is usually mild and asymptomatic.

8. Cases occurring in late adolescence are often related to eating disorders.

9. With aging, hypophosphatemia is often related to alcoholism, tumors, malabsorption, or vitamin D deficiency

10. Oncogenic Osteomalacia,

11. History of long-standing alcohol use and chronic malnutrition.

12. Receiving parenteral nutrition with inadequate quantities of phosphate replacement.

13. Associted multiple myeloma or other paraproteinemia

14. Exposure to heavy metals and paraproteinemias.

15. Drugs that can produce renal phosphate wasting include loop diuretics, cisplatinum, pamidronate, acetazolamide, and glucocorticoids,

16. Treatment of diabetic ketoacidosis

17. Extensive burns

18. Use of growth factors

19. Bone marrow transplant

20. ICU setting

21. People with eating disorders or dietary deficiencies due to socioeconomic, dental, or swallowing difficulties may also become hypophosphatemic when fed an adequate diet.

22. Malabsorption of intestinal phosphate

23. Primary intestinal disorders, such as Crohn disease or celiac sprue, can limit phosphate absorption, leading to hypophosphatemia.

24. Forced saline diuresis. Extracellular volume expansion or administration of bicarbonate can cause loss of phosphate through the kidney

25. Vitamin D deficiency. Simple vitamin D deficiency results in hypophosphatemia, at least in part from renal wasting. Vitamin D deficiency can result from several mechanisms: poor oral intake, lack of sun exposure, drug-induced hypermetabolism of vitamin D precursors in the liver, or loss of vitamin D binding protein in the urine in nephrotic syndrome.

26. Phosphate wasting can result from genetic or acquired renal disorders X-linked hypophosphatemic, Autosomal dominant hypophosphatemic rickets, Hereditary hypophosphatemic rickets with hypercalciuria, Vitamin D-resistant rickets is an autosomal recessive disorder

27. Acute respiratory alkalosis or hyperventilation produces hypophosphatemia by stimulating a shift of phosphate into the cells. This mechanism is responsible for the hypophosphatemia observed with salicylate overdose, panic attacks, and sepsis.

28. Insulin increases cell phosphate uptake and causes hypophosphatemia during treatment of diabetic ketoacidosis, refeeding, and parenteral nutrition therapy.

29. Exogenous epinephrine also stimulates cellular phosphate uptake.

30. Several cytokines reportedly stimulate intracellular phosphate shift. This mechanism is perhaps responsible for the hypophosphatemia observed in the ICU setting of trauma, extensive burns, and bone marrow transplantation.

31. In the “hungry-bone” syndrome, there is rapid uptake of phosphate into bone after the initial treatment of osteomalacia or rickets or postparathyroidectomy.

32. Others causes not mentioned here.

The Effects of Phosphate Depletion:

1. Hypophosphatemia can lead to hypomagnesemia (81).

2. It is theoretically possible that phosphate depletion is associated with a rise in cytosolic calcium, and such an increment contributes significantly to the decrease in ATP content of cells through an effect on mitochondrial oxidation and/or phosphorylation. Indeed, several studies have demonstrated that phosphate depletion is associated with a significant rise in basal levels of intracellular Ca in brain synaptosomes, pancreatic islets, and polymorphonuclear leukocytes. This derangement's lead to a rise in intracellular Ca, an event that inhibits mitochondrial oxidation and phosphorylation and hence reduces ATP production. Thus the available data are consistent with the notion that calcium entry into cells is increased and calcium extrusion out of the cells is decreased in phosphate depletion. (83)

3. Phosphate depletion affects the phospholipid metabolism of cells. Indeed, it has been shown that phosphate depletion causes significant decrements in total phospholipid content and in the contents of phosphatidylinositol, phosphatidylserine, and phosphatidylethanolamine of brain synaptosomes. These changes can render the cell membrane more permeable to Ca and lead to augmented entry of this ion into the cells. (83)

4. The fall in ATP content of cells in phosphate depletion is not only due to the phosphorus deficiency per se, but also to the increase in intracellular Ca. (83)

5. Also alterations in phospholipid metabolism of cell membrane adversely affect the activity of Na—K-ATPase and therefore also contributes to reduced calcium extrusion out of the cell and to rise in intracellular Ca. Indeed, studies in brain synaptosomes, in pancreatic islets, and in cardiac myocytes have shown that the Vmax of Ca-ATPASE AND Na—K-ATPase are reduced in phosphate depletion. (83)

6. One of the hallmarks of hypophosphatemia and cellular phosphate depletion is the striking increase in urinary excretion of calcium and magnesium. Magnesium excretion may be sufficiently large to lead to overt hypomagnesemia. (85)

7. Phosphate depletion decreases magnesium entry in isolated distal cells, which in turn leads to diminished epithelial magnesium transport. Hypophosphatemia, hypokalemia, and metabolic acidosis may cause renal magnesium wasting that may be severe enough to lead to hypomagnesemia. (85).

8. Phosphate depletion can be associated with hypomagnesemia (81, 85, 87) with decrease Mg reabsorption in distal tubule (76) and decrease Mg entry in isolated distal cells (85).

### Result of Increase Intracellular Phosphate is Decrease Risk of Cancer (93, 94, and 95)

### B. Increase in Intracellular Calcium

This can also occur due to other causes, other than decrease intracellular ATP, Cancer might happen without these but their presence might accelerate the process. These include

1. Extra cellular application of ATP (180)

2. Mechanical stimulation (180,181) and cell injury (65, 50, 77)

3. Histamine (79).

4. Muscarinic Agonists (58).

5. Membrane depolarization (110,154)

6. Glucose and Deoxyglucose (112,114) Crabtree effect.

7. Increase Serum Ca (55,56) which relate to increase intake which is associated with increase cancer prostate (63, 92, 93)

8. Chemicals, like Carbachol (79,166), TEA, 4-AP (158), Ionomycin, PHA (176), CCCP (114).

9. Phosphate Depletion which affect cell membrane lead to increase intracellular Ca (83).

10. Abnormal permeability of Ca-ion channel (77).

11. Hypotonic solution (124) leads to cell swelling and membrane depolarization which result in increase Ca entry.

The Effects of Increase Intracellular Calcium Include:

1. Cell membrane refractory to depolarization which lead to loss of cell membrane excitability leads to defective cell response (56, 83).

2. Decrease or closure gap junctions permeability (58, 77, 78, 103).

3. Uncontrolled enzyme activation (77, 90).

4. Increase ACTH (154), neurosecretion, protein secretion and exocytosis (90,112).

5. Ca²⁺ increases cell aerobic metabolism by activating several mitochondrial primary dehydrogenases. In addition to activation of mitochondrial dehydrogenases, Ca²⁺ also produces inhibition of F₁F₀-ATP synthase, the latter effect presumably being more pronounced than the former. The concerted action of the two mechanisms results in a considerable increase in mitochondrial membrane potential and redox state of nicotinamide nucleotides, which are very important for biosynthetic processes in these highly proliferating cells. (114).

6. Association of inhibitory unites with F1-F0 ATPase complex leads to inhibition of coupled respiration leads to decrease ATP (83, 114, 118).

7. Activate large K channel, CL channel, nonspecific cation channels which leads to increase intracellular Na and decrease intracellular CL and K (58, 122, 189).

### The Inhibition of Increase Intracellular Calcium is associated with Antiproliferative Effect and Inhibition of Tumor Growth (165, 166, 125)

The Effects of This Decrease in Cell Energy will Include

1. Decrease in Ca pump which will lead to decrease Ca extrusion which will lead to increase in intracellular Ca (83). Discussed above.

2. Decrease in Na—K pump (83,97) which will lead to loss of k (120) and increase entry of Na with possible cotransport of sugars and amino acids and exchange with Mg i(102).

3. Activation of K ATP channels

This can also be caused by other causes, besides decrease Intracellular ATP, Cancer might happen without these but their presence might accelerate the process.

1. K channel openers (1)

2. Depolarization (183,201)

3. Increase cellular ADP and low NAD, NADH, NADP AND NADPH (204).

4. Hypo osmotic swelling (129)

5. LTD4(129)

6. Free Radicals (109,167) which leads to cell depolarization.

7. Increase intracellular Ca (187, 189, 197, 208, 209)

The Effects of Activation of KATP Channels.

1. Decrease gap junction permeability (1).

2. Increase cellular DNA synthesis (115).

3. Membrane hyperpolarization (146) which inhibit communications through gap junctions (1).

4. Potassium loss (1, 119, 120, 146, 187).

Inhibition of KATP channels leads to arrest cells in G0/G1 of the cell cycle and inhibition of cell proliferation and attenuate DNA synthesis (115, 155, 168, 173, 137). The inhibition can be by ATP and leads to increase gap junction permeability (1, 119, 187, 196, 197, 205, 206, 209).

### 4. Decreased in Gap Junction Permeability

This can also be caused by other causes, besides decrease Intracellular ATP, Cancer might happen without these but their presence might accelerate the process.

1. Chemical tumor promoters like Phorbol esters, DDT (26), Phenobarbital, Unsaturated Fatty acids, saccharin (4,27)

2. Various oncogenes: Ras, Raf, Neu, Src, Mos (4, 24, 35).

3. Polyaromatic hydrocarbons (14, 17, 22, 23), Naphthalenes, Crude oil products (23), Cigarette smoke (23), H2O2 (21, 37, 38) which leads to hyperphosphorylation of connexins, Breast tumor promoter: heptachlor and heptachlor epoxid (25), halogenated hydrocarbons (26), n-3 Fatty acids-corragreenan (36,41), ACN (13), Alpha-linolenate and Linoleic acid (20,37), TPA (21, 27, 33, 38).

4. Defect in post translation phosphorylation of cx43 or its knock out mutation (4, 29, 31).

5. Increase intracellular Ca(1)

6. PKC leads to hyperphosphorylation of GJIC(1, 21, 37, 112)

7. Membrane hyperpolarization (1).

Effects of decreased Gap Junctions Permeability.

1. Allows factors that control intracellular events to exceed a critical mass necessary for the cell to either proliferate or undergo apoptosis (17).

2. Release of a cell from growth suppression leads to phosphorylation of ERKi, ERK2 (mapk activation) leads to mitogenic events (14)

3. Stable abnormal regulation of gap junction has been associated with the activation of several oncogenes (28,30)

4. It is associated with Astrocyte proliferation (42).

5. Leads to increase glucose uptake which leads to increase ribose-5-phosphate through pentose phosphate pathway which leads to synthesis of nucleic acids (42).

Increase in Gap Junctions Permeability will Reduce Tumorigenicity.

Antitumor promoters and antioncogene drugs can upregulate Gap Junction permeability. (4, 5, 7, 10, 18, 40)

### 5. Magnesium Depletion

is associated with Hypophosphatemia (81, 85, 87) and Inhibition of Na (+)-K+ ATPase results in intracellular Mg ++ deficiency (101). This can also be caused by other causes, Cancer might happen without these but their presence might accelerate the process.

It is also caused by

1. Malabsorption of magnesium in the ileum results in hypomagnesemia. Situations of decreased absorption include malabsorption syndromes (eg, celiac sprue), radiation injury to the bowel, bowel resection, or small bowel bypass

2. chronic diarrhea, laxative abuse, inflammatory bowel disease, or neoplasm.

3. low dietary intake of magnesium

4. Alcoholics are classically hypomagnesemic in part due to poor nutrition.

5. Diabetic patients who are not receiving magnesium supplements may have dietary deficiencies in magnesium.

6. Renal losses from primary renal disorders or secondary causes (eg, drugs, hormones, osmotic load)

7. Primary renal disorders cause hypomagnesemia by decreased tubular reabsorption of magnesium by the damaged kidneys. This condition occurs in the diuretic phase of acute tubular necrosis, postobstructive diuresis, and renal tubular acidosis.

8. Drugs may cause magnesium wasting.

A. Diuretics (eg, thiazide, loop diuretics) decrease the renal threshold for magnesium reabsorption in addition to wasting of potassium and calcium.

B. Cisplatin causes dose-dependent kidney damage in 100% of patients receiving this drug.

C. Pentamidine and some antibiotics also cause renal magnesium wasting.

D. Fluoride poisoning similarly causes hypomagnesemia.

9. Endocrine disorders may cause hypomagnesemia.

A. Primary aldosteronism decreases magnesium levels by increasing renal flow.

B. Hypoparathyroidism and hyperthyroidism may cause renal wasting.

10. Osmotic diuresis results in magnesium loss in the kidney. Alcoholics become hypomagnesemic partially by an osmotic diuresis from alcohol. Urinary losses have been reported to be 2-3 times control values.

11. Extracellular volume expansion, as in cirrhosis or intravenous (IV) fluid administration, may decrease magnesium levels.

12. Redistribution of magnesium into cells may cause lower magnesium levels. Insulin causes this effect.

13. Excessive lactation may create a significant amount of magnesium loss.

14. Hungry bone syndrome may lead to lower serum magnesium concentrations.

15. Pregnant women have been found to be magnesium depleted, especially those women who experience preterm labor.

Effects of Magnesium Depletion.

1. During magnesium depletion, intracellular potassium falls, and the ability of the kidney to conserve potassium is impaired with the subsequent development of hypokalemia and a total body potassium deficit. (87, 96).

2. Chronic magnesium and zinc deficiency seems to be associated with the development of ALL and malignant lymphoma in a group of patients (99).

3. Magnesium level below 12.75 micrograms/ml in cancer patients increased the risk of cancer metastases to the liver (100).

4. ATP in the presence of Mg2+ appears to be required to maintain the SUR2B/K ir6.1 channels in an operational state, but ATP, at physiological concentrations, does not inhibit their activity significantly. (119) the SUR2B/K ir6.1 channels partially inhibited by ATP are stimulated by ADP in the presence of Mg2+. (119) a significant fraction of ATP is liganded at the intracellular free Mg2+ concentration. Findlay, for example, noted that ATP4-inhibits channel activity, but that application of the same total concentration of ATP with magnesium enhances inhibition (119).

When K atp channels are put into ATP-free solution they open, but then rundown or lose their activity, which can be restored or refreshed by a brief application of mM concentrations of MgATP. This process can be likened to switching the channel from a nonoperational to an operational state (119). Mg is important for K atp channels activity. Extracellular perfusion of 5 mM Mg2+ dramatically slowed the activation of The outward rectifier current (IK). (121)

The Effects of Correction of Decreased Intracellular Magnesium Depletion.

Interleukin 2 (IL-2) can cause partial or complete tumor regression in approximately 20% of patients with renal cell carcinoma. During IL-2 therapy, lymphocyte Mg increases coincident with serum Mg depletion (101)

### 6. Potassium Depletion.

is associated with Hypomagnesemia. (87,96), Phosphate depletion and decrease the activity of Na—K-ATPase (83). During magnesium depletion, intracellular potassium falls, and the ability of the kidney to conserve potassium is impaired with the subsequent development of hypokalemia and a total body potassium deficit. (87, 96) Hypokalemia has been estimated to occur in approximately 75% of all patients with malignancy at some time during their illness. (96). Hypokalemia was the most frequent electrolyte abnormality observed in 41 patients (63%) namely in 34 patients with AML and 7 with ALL (98).

Causes of Potassium Depletion

This can also be caused by other causes, Cancer might happen without these but their presence might accelerate the process.

1. Deficient intake. Poor potassium intake alone is an uncommon cause of hypokalemia but occasionally can be seen in

A. very elderly individuals unable to cook for themselves or unable to chew or swallow well. Over time, such individuals can accumulate a significant potassium deficit. Another clinical situation where hypokalemia may occur due to poor intake is in

B. patients receiving total parenteral nutrition (TPN), where potassium supplementation may be inadequate for a prolonged period of time.

C. Eating disorders: Anorexia, bulimia, starvation, pica, and alcoholism

D. Dental problems: Inability to chew or swallow

E. Poverty: Lack of food, ie, “tea-and-toast” diet of elderly individuals.

2. Increased excretion of potassium, especially coupled with poor intake, is the most common cause of hypokalemia.

A. Renal potassium losses. The most common mechanisms leading to increased renal potassium losses include enhanced sodium delivery to the collecting duct, as with diuretics (carbonic anhydrase inhibitors, loop diuretics, thiazide diuretics). Occult diuretic use is far more common than either congenital tubular disorder and is, in fact, also called “pseudo Bartter.”; mineralocorticoid excess, as with primary or secondary hyperaldosteronism; or increased urine flow and polyuria, as with an osmotic diuresis, Mannitol and hyperglycemia can cause osmotic diuresis.

B. Gastrointestinal losses, most commonly from diarrhea, also are common causes of hypokalemia. Vomiting is a common cause of hypokalemia. Vomiting produces volume depletion and metabolic alkalosis. These 2 processes are accompanied by increased renal potassium excretion. Volume depletion occurs through the activation of secondary hyperaldosteronism, which, in turn, leads to enhanced cortical collecting tubule secretion of potassium in response to enhanced sodium reabsorption.

C. Metabolic alkalosis also increases collecting tubule potassium secretion due to the decreased availability of hydrogen ions for secretion in response to sodium reabsorption.

D. Hyperaldosteronism due to licorice ingestion leads to hypertension (glycyrrhizic acid in some types of licorice has mineralocorticoid effects).

E. occult laxative use, diuretic use, bulimia, or one of the unusual tubular disorders such as Bartter syndrome or Gitelman syndrome.

F. Endogenous mineralocorticoid excess Cushing disease, Primary hyperaldosteronism, most commonly due to adenoma or bilateral adrenal hyperplasia

G. Secondary hyperaldosteronism due to volume depletion, congestive heart failure, cirrhosis, or vomiting

H. Adrenocortical carcinoma, Tumor that is producing adrenocorticotropic hormone

I. Congenital disorders.

J. Hyperreninism due to renal artery stenosis

K. Exogenous mineralocorticoid excess

L. Steroid therapy for immunosuppression

M. Renal tubular disorders—Type I and type II renal tubular acidosis

N. Exogenous bicarbonate ingestion

O. Amphotericin B, Gentamicin

P. obligatory renal losses are 10-15 mEq/d. Thus, chronic losses occur in the absence of any ingested potassium

Q. glucagon impairs potassium entry into cells.

R. An acute increase in osmolality causes potassium to exit from cells.

S. An acute cell/tissue breakdown releases potassium into extracellular space

3. The third is due to a shift from extracellular to intracellular space. This pathogenetic mechanism also often accompanies increased excretion, leading to a potentiation of the hypokalemic effect of excessive loss. Intracellular shifts of potassium often are episodic and frequently are self-limited, for example, with acute insulin therapy for hyperglycemia.

Shift of potassium into the intracellular space may occur due to the following:

Recurrent episodes of paralysis

Use of high doses of insulin

High-dose beta agonist therapy (eg, for chronic obstructive pulmonary disease)

Alkalosis, metabolic or respiratory

Insulin administration or glucose administration: This stimulates insulin release.

Intensive beta-adrenergic stimulation

Hypokalemic periodic paralysis

Thyrotoxic periodic paralysis

Adrenergic stimuli: (1) Beta-adrenergic stimuli enhance potassium entry into cells, and (2) alpha-adrenergic stimuli impair potassium entry into cells.

Refeeding: This is observed in prolonged starvation, eating disorders, and alcoholism

4. Other causes: 21% of hospitalized patients have serum potassium levels lower than 3.5 mEq/L, with 5% of patients achieving potassium levels lower than 3 mEq/L.

5. Groups with a high incidence of hypokalemia include (1) individuals with eating disorders, regarding which one series by Greenfeld et al reported a 4.6% incidence of hypokalemia in an outpatient setting, and (2) patients with AIDS, of which 23.1% of hospitalized patients are hypokalemic. (3) African Americans and females are more susceptible. Risk is enhanced by concomitant illness such as heart failure or nephrotic syndrome. (4) Congenital disorders: Bartter syndrome, Gitelman syndrome, Liddle syndrome.

Effects of Decrease Intracellular Potassium.

Since decrease intracellular k occur with K channels openers, the effects should be close to effects of activation of K ATP Channels.

The Results from Increasing Potassium.

1. When the cells were bathed in symmetrical high-K+ solution, Ip also completely disappeared. a transient outward current (peak current, Ip)(122). The resulting reduction in K⁺ion efflux leads to the disruption of the chain of biochemical processes required for LNCaP cell proliferation (125).

2. RVD was blocked by high K(+),(141). In all of these conditions, we observed a close correspondence between the rate of proliferation and the mean cell volume. The proliferation decreased when volume increased(127). Proliferation was fully inhibited when cell volume was increased by 25%. (147). ). blockers of net K efflux through K channels (e.g. isotonic KCl or 20 mM TEA); prevent RVD(152).

3. Increased extracellular K+ concentration inhibited tumour cell growth in a dose-related fashion in both cell lines, Two human brain tumour cell lines, U-373 MG astrocytoma and SK-N-MC neuroblastoma,.

4. Agonist (carbachol or serum)-induced intracellular Ca2+ mobilization was also blocked by the pretreatment of growth-inhibitory concentrations of K+ channel modulators and high extracellular K+. (166).

The Treatment.

1. Phosphate to correct phosphate depletion (83).

For severe hypophosphatemia (<1 mg/dL), parenteral preparations of phosphate should be used for repletion.

For less severe hypophosphatemia (1-2 mg/dL), PO phosphate salt preparations can be used.

Neutra-Phos packets contain 250 mg of phosphorus/packet. Tablets contain either 250, 125.6, or 114 mg apiece.

The liquid preparations are available as 250 mg/75 mL.

Adult Dose Initial dose: 0.1 mmol/kg of K 2 PO 4 or Na 2 PO 4 q6h IV (32 mmol/d)

Aggressive IV replacement: 0.2-0.3 mmol/kg of K 2 PO 4 or Na 2 PO 4 over 6 h

For oral replacement, 250 mg as capsule, liquid, or packet tid/qid is generally adequate.

Pediatric Dose 0.25-0.5 mmol/kg PO over 4-6 h; repeat if symptomatic hypophosphatemia persists

The Goal is to Keep the Phosphorus Level between 4-5mg/dL

2. Magnesium.

Oral supplementation should be given when patient is mildly depleted of magnesium (ie, magnesium level >1 mEq/L and asymptomatic).

Other oral supplements (eg, magnesium oxide, magnesium hydroxide) may be used. Oral supplementation should be considered in patients who do not have a correctable cause for their hypomagnesemia.

Adult Dose 500 mg (27 mg elemental magnesium) PO qd Magnesium sulfate—Supplementation via IV infusion should be given to patients with moderately severe to severe depletion.

Adult Dose 2-4 g of 50% magnesium sulfate (16.6-33.3 mEq) diluted in saline or dextrose IV over 30-60 min

In cases of life-threatening arrhythmias, give same amount IV push

Pediatric Dose 1 mEq/kg IV on day 1; 0.5 mEq/kg/d over next 3 d

Pediatric Dose 3-6 mg elemental magnesium/kg/d PO divided tid/qid; not to exceed 400 mg in 24 h

The Goal is to Keep the Magnesium Level between 2.5-3mg/dL

3. Potassium.

The first step is to identify and stop ongoing losses of potassium.

Repletion of potassium losses is the second step.

Repletion magnesium if low.

Potassium citrate (Urocit K, Polycitra, Bicitra)—Oral preparation with a base instead of an acid anion. Generally used for patients who form calcium stones or for severe metabolic acidosis.

Adult Dose Urocit: 3 tab PO tid

Polycitra or Bicitra: 1 mL/kg/d

Pediatric Dose Urocit: Not established

Polycitra or Bicitra: Administer as in adults

Potassium gluconate

adults iv 10-40 mEq/2-3 hrs.

50-100 mEq/day on 1, 2, 3 doses.

Pediatrics 0.5-1 mEq/kg not more than 30-40 mEq/dose.not to exceed 0.3-0.5/kg/hr.

2-4 mEq/kg/d in divided doses.

The Goal is to Keep the Potassium Level between 4-5 mg/dL

4. Fructose if available or glucose. Fructose may protect against prostate cancer by causing a rapid shift of phosphate from the extracellular to intracellular compartment and as a source of energy. (93, 94, 95).

5. Verapamil. (Verelan PM) 200-400 mg as tolerated.

As a Ca channel blocker with its antiproliferative ability and by its inhibition of K⁺channels. (165, 166, 125). It will help to decrease Crabtree effect from glucose.

6. Alkalosis to help move Phosphorus, Potassium, magnesium to the cell.

### Refferences

1. Velasco A, Tobernero A, Grando B, Medina J. ATP-Sensitive Potassium Channel Regulates Astrocytic Gap Junction Permeability by a Ca-Independent Mechanism. Journal of neurochemistry, Vol. 74, No.3, 2000 1249-1256.

2. Vera B et al, Inhibition of astrocyte gop junctional communication by ATP depletion is reversed by calcium sequestration. FEBS Lett 1996 Sep. 2; 392(3):225-8.

3. Mark A, et al. Ciliary Neurotropic factor (CNTF) in combination with its soluble receptor (CNTFRα) increase connexin43 expression and suppresses Growth of C6 Glioma Cells. Cancer research 62,3544-3548.

4. Trosko J E, Chang C C. Mechanism of up-regulated gap junctional intercellular communication during chemoprevention and chemotherapy of cancer. Mutat Res 2001 Sep. 1;480-481:219-29

5. Trosko J E, Chang C C. Modulotion of cell-cell communication in the cause and chemoprevention/chemotherapy of cancer. Biofactors 2000; 12(1-4):259-63

6. Trosko J E, Chang C C. Role of stem cells and gap junctional intercellulor communication in human cardnogenesis. Radiat Res 2001 January;155(1 Pt 2):175-180

7. Sai K, Kanno J, Hasegawa R, Trosko J E, Inoue T.Prevention of the down-regulation of gap junctional intercellular communication by green tea in the liver of mice fed pentachlorophenol. Carcinogenesis 2000 September;21(9):1671-6

8. Na H K, Wilson MR, Kong K S, Chang C C, Grunberger D, Trosko J E. Restoration of gap junctional intercellular communication by caffeic acid phenethyl ester (CAPE) in a ras-transformed rat liver epithelial cell line. Cancer Lett 2000 Aug. 31; 157(1):31-8

9. Suzuki J, Na H K, Upham B L, Chang C C, Trosko J E.Lambda-carrageenan-induced inhibition of gap-junctional intercellular communication in rat liver epithelial cells. Nutr Cancer 2000;36(1):122-8

10. Kang K S, Kang B C, Lee B J, Che J H, Li G X, Trosko J E, Lee Y S. Preventive effect of epicatechin and ginsenoside Rb (2) on the inhibition of gap junctional intercellular communication by TPA and H(2)O(2). Cancer Lett 2000 Apr. 28; 152(1):97-106

11. Trosko J E, Chang C C, Wilson M R, Upham B, Hayashi T, Wade M. Gap junctions and the regulation of cellular functions of stem cells during development and differentiation. Methods 2000 February;20(2):245-64

12. Ogawo T, Hayashi T, Kyoizumi S, Ito T, Trosko J E, Yorioka Up-regulation of gap junctional intercellular communication by hexamethylene bisacetamide in cultured human peritoneal mesotheliol cells. Lab Invest 1999 December;79(12):1511-20

13. Komendulis L M, Jiang J, Zhang H, deFeijter-Rupp H, Trosko J E, Klounig J E. The effect of acrylonitrite on gap junctional intercellular communication in rat astrocytes. Cell Biot Toxicol 1999 June;15(3):173-83

14. Rummet A M, Trosko J E, Wilson M R, Upham B L. Polycyclic aromatic hydrocarbons with bay-like regions inhibited gap junctional intercellular communication and stimulated MAPK activity. Toxicol Sci 1999 June;49(2):232-40

15. Trosko J E, Chang C C, Upham B, Wilson M.Epigenetic toxicology as toxicant-induced changes in intracellular signalling leading to altered gap junctional intercellular communication. Toxicol Lett 1998 Dec. 28;102-103:71-8

16. Upham B L, Deocampo N D, Wurl B, Trosko J E. Inhibition of gap junctional intercellular communication by perfluorinated fatty acids is dependent on the chain length of the fluorinated tail. Int J Cancer 1998 Nov. 9;78(4):491-5

17. Upham B L, Weis L M, Trosko J E Modulated gap junctional intercellular communication as a biomorker of PAH epigenetic toxicity: structure-function relationship. Environ Health Perspect 1998 August;106 Suppl 4:975-81

18. de Feijter-Rupp H L, Hayashi T, Kalimi G H, Edwards P, Redpoth J L, Chang C C, Stanbridge E J, Trosko J E. Restored gap junctional communication in non-tumorigenic HeLa-normal human fibroblast hybrids. Carcinogenesis 1998 May;19(5):747-54

19. Trosko J E, Ruch R J. Cell-cell communication in cardnogenesis. Front Biosci 1998 Feb. 15;3:D208-36

20. Hayashi T, Matesic D F, Nomata K, Kang K S, Chang C C, Trosko J E. Stimulation of cell proliferation and inhibition of gap junctional intercellular communication by linoleic acid. Cancer Lett 1997 Jan. 15;112(1):103-11

21. Upham B L, Kong K S, Cho H Y, Trosko J E. Hydrogen peroxide inhibits gap junctional intercellular communication in glutathione sufficient but not glutathione deficient cells. Carcinogenesis 1997 January;18(1):37-42

22. Weis L M, Rummel A M, Masten S J, Trosko J E, Upham B L Bay or baylike regions of polycyclic aromatic hydrocarbons were potent inhibitors of Gap junctional intercellular communication. Environ Health Perspect 1998 January;106(1):17-22

23. Upham B L, Weis L M, Rummel A M, Masten S J, Trosko J E. The effects of anthracene and methylated anthracenes on gap junctional intercellular communication in rat liver epithelial cells. Fundam Appl Toxicol 1996 December;34(2):260-4

24. Hofer A, Saez J C, Chang C C, Trosko J E, Spray D C, Dermietzel R. C-erbB2/neu transfection induces gap junctional communication incompetence in glial cells. J Neurosci 1996 Jul. 15; 16(14):4311-21

25. Nomata K, Kang K S, Hayashi T, Matesic D, Lockwood L, Chang C C, Trosko J E. Inhibition of gap junctional intercellular communication in heptachlor- and heptachlor epoxide-treated normal human breast epithelial cells. Cell Biol Toxicol 1996 April;12(2):69-78

26. Kong K S, Wilson M R, Hoyashi T, Chang C C, Trosko J E. Inhibition of gap junctional intercellular communication in normal human breast epithelial cells after treatment with pesticides, PCBs, and PBBS, alone or in mixtures. Environ Health Perspect 1996 February;104(2):192-200

27. Chaudhuri R, Sigler K, Dupont E, Trosko J E, Malkinson A M, Ruch R J. Gap junctional intercellular communication in mouse lung epithelial cell lines: effects of cell transformation and tumor promoters. Cancer Lett 1993 Jul. 30;71(1-3):11-8

28. Trosko J E, Chang C C, Madhukar B V. The role of modulated gap junctional intercellular communication in epigenetic toxicology. Risk Anal 1994 June;14(3):303-12

29. Oh S Y, Dupont E, Madhukar B V, Briand J P, Chang C C, Beyer E, Trosko J E. Characterization of gap junctional communication-deficient mutants of a rat liver epithelial cell line. Eur J Cell Biol 1993 April;60(2):250-5

30. Trosko J E, Madhukar B V, Chong C C. Endogenous and exogenous modulation of gap junctional intercellular communication: toxicological and pharmacological implications. Life Sd 1993;53(1):1-19

31. Jou Y S, Matesic D, Dupont E, Lu S C, Rupp H L, Madhukar B V, Oh S Y, Trosko J E, Chang C C. Restoration of gap-junctional intercellular communication in a communication-deficient rat liver cell mutant by transfection with connexin 43 cDNA. Mol Carcinog 1993;8(4):234-44

32. Trosko J E, Chang C C, Madhukar B V, Klaunig J E. Chemical, oncogene and growth factor inhibition gap junctional intercellular communication: an integrative hypothesis of carcinogenesis. Pathobiology 1990;58(5):265-78

33. Ruch R J, Trosko J E, Madhukar B V. Inhibition of connexin43 gap junctional intercellular communication by TPA requires ERK activation. J Cell Biochem 2001 Jun. 26-Jul. 25;83(1):163-9

34. Ruch R J, Madhukar B V, Trosko J E, Klaunig J E. Reversal of ras-induced inhibition of gap-junctional intercellular communication, transformation, and tumorigenesis by lovastatin. Mol Carcinog 1993;7(1):50-9

35. Jou Y S, Layhe B, Matesic D F, Chang C C, de Feijter A W, Lockwood L, Welsch C W, Klaunig J E, Trosko J E. Inhibition of gap junctional intercellular communication and malignant transformation of rat liver epithelial cells by neu oncogene. Carcinogenesis 1995 February;16(2):311-7

36. Hasler C M, Trosko J E, Bennink M R. Incorporation of n-3 fatty acids into WB-F344 cell phospholipids inhibits gap junctional intercellular communication. Lipids 1991 October;26(10):788-92

37. Hasler C M, Bennink M R, Trosko J E. Inhibition of gap junction-mediated intercellular communication by alpha-linolenate. Am J Physiol 1991 July;261(1 Pt 1):C161-8

38. Hasler C M, Frick M A, Bennink M R, Trosko J E. TPA-induced Inhibition of gap junctional intercellular communication is not mediated through free radicals. Toxicol Appi Pharmacol 1990 May;103(3):389-98

39. Trosko J E, Chang C C Role of stem cells and gap junctional intercellular communication in human carcinogenesis. Radiat Res 2001 January;155(1 Pt 2):175-180

40. Na H K, Wilson M R, Kang K S, Chang C C, Grunberger D, Trosko J E.Restoration of gap junctional intercellular communication by caffeic acid phenethyl ester (CAPE) in a ras-transformed rat liver epithelial cell line. Cancer Lett 2000 Aug. 31; 157(1):31-8

41. Suzuki J, Na H K, Upham B L, Chang C C, Trosko J E.Lambda-carrageenan-induced inhibition of gap-junctional intercellular communication in rat liver epithelial cells. Nutr Cancer 2000;36(1):122-8

42. Tabernero A, Jimenez C, Velasco A, Giaume C, Medina J M. The enhancement of glucose uptake caused by the collapse of gap junction communication is due to an increase in astrocyte proliferation. Source Journal of Neurochemistry. 78(4):890-8, 2001 August

43. Pathophysiology 4^{th},ed. The Biological Basis for Disease in Adults & Children. P.306

44. Pathophysiology 4^{th},ed. The Biological Basis for Disease in Adults & Children P 306-307.

45. Pathophysiology 4^{th},ed. The Biological Basis for Disease in Adults & Children P 308.

46. Pathophysiology 4^{th},ed. The Biological Basis for Disease in Adults & Children P308

47. Pathophysiology 4^{th},ed. The Biological Basis for Disease in Adults & Children P309.

48. Pathophysiology 4^{th},ed. The Biological Basis for Disease in Adults & Children P 315

49. Pathophysiology 4^{th},ed. The Biological Basis for Disease in Adults & Children P320

50. Pathophysiology 4^{th},ed. The Biological Basis for Disease in Adults & Children P 62 path.

51. Pathophysiology 4^{th},ed. The Biological Basis for Disease in Adults & Children P63 path.

52. Pathophysiology 4^{th},ed. The Biological Basis for Disease in Adults & Children P67 path.

53. Pathophysiology 4^{th},ed. The Biological Basisfor Disease in Adults & Children P51 path.

54. Pathophysiology 4^{th},ed. The Biological Basis for Disease in Adults & Children P52 path.

55. Pathophysiology 4^{th},ed. The Biological Basis for Disease in Adults & Children P 98, p96, P100.

56 Pathophysiology 4^{th},ed. The Biological Basis for Disease in Adults & Children P100 path.

57. Nilius B, Droogmans G. Ion channels and their functional role in vascular endothelium. Physiol Rev 2001 October;81(4):1415-59

58. Kallenberg L A. Calcium signalling in secretory cells. Arch Physiol Biochem 2000 December;108(5):385-90

59. Dupont G, Swillens S, Clair C, Tordjmann T, Combettes L Hierarchical organization of calcium signals in hepatocytes: from experiments to models. Biochim Biophys Acta 2000 Dec. 20;1498(2-3):134- 52

60. Martins-Ferreira H, Nedergoard M, Nicholson C. Perspectives on spreading depression. Brain Res Brain Res Rev 2000 April;32(1):215-34

61. Pathophysiology 4^{th}, ed. The Biological Basisfor Disease in Adults & Childre P 105 path.

62. P11 by Eleanor review.

63. Green J, et al. Acute phosphate depletion inhibits the Na+/H+ antiporter in a cultural renal cell line. Am J Physiol 1993 September;265(3 pt 2 ):F440-8.

64. Gasbarrini A, Borle A B, Farghali H, Francavilla A, Van Thiel D. Fructose protects rat hepatocytes from anoxic injury. Effect on intracellular ATP, Ca2+i, Mg2+i, Na+i, and pHi. J Biol Chem 1992 Apr. 15;267(11):7545-52

65. Gasbarrini A, Borle A B, Farghali H, Bender C, Francavilla A, Van Thiel D.Effect of anoxia on intracellular ATP, Na+i, Ca2+i, Mg2+i, and cytotoxicity in rat hepatocytes. J Biol Chem 1992 Jun. 25;267(18):13114

66. Nakayama S, Nomura H, Tomita T. Intracellular-free magnesium in the smooth muscle of guinea pig taenia caeci: a concomitant analysis for magnesium and pH upon sodium removal. J Gen Physiol 1994 May;103(5):833-51

67. Li H Y, Dai L J, Quamme G A. Effect of chemical hypoxia on intracellular ATP and cytosolic Mg2+ levels. J Lab Clin Med 1993 September;122(3):260-72

68. Li H Y, Quamme G A. Caffeine decreases intracellular free Mg2+ in isolated adult rat ventricular myocytes. Biochim Biophys Acta 1997 Jan. 10;1355(1):61-8

69. Crosby V. Wilcock A. Corcoran R e. the safety and efficacy of a single dose(500 mg or 1 g) of intravenous magnesium sulfate in neuropathic pain poorly responsive to strong opioid analgesics in patients with cancer. [Clinical Trial. Journal Article] Journal of Pain & Symptom Management. 19(1):35-9, 2000 January

70. Li H Y, Dai L J, Krieger C, Quamme G A.lntracellular Mg2+ concentrations following metabolic inhibition in opossum kidney cells. Biochim Biophys Acta 1993 Jun. 19;1181(3):307-15

71. Altura B M, Zou L Y, Altura B T, Jelicks L, Wittenberg B A, Gupta R K. Beneficial vs. detrimental actions of ethanol on heart and coronary vascular muscle: roles of Mg2+ and Ca2+. Alcohol 1996 September-October;13(5):499-513

72. Altura B M, Gebrewold A, Zhang A, Altura B T, Gupta R K. Magnesium deficiency exacerbates brain injury and stroke mortality induced by alcohol: a 31P-NMR in vivo study. Alcohol 1998 April;15(3):181-3

73. Altura B M, Gebrewold A, Zhang A, Altura B T, Gupta R K. Short-term reduction in dietary intake of magnesium causes deficits in brain intracellular free Mg2+ and [H+]i but not high-energy phosphates as observed by in vivo 31P-NMR. Biochim Biophys Acta 1997 Aug. 21;1358(1):1-5

74. Altura B M, Gebrewold A, Altura B T, Gupta R K. Role of brain [Mg2+]i in alcohol-induced hemorrhagic stroke in a rat model: a 31P-NMR in vivO study. Alcohol 1995 March-April;12(2):131-6

75. Altura B M, Altura B T, Carella A, Gebrewold A, Murakawa T, Nishio A. Mg2+-Ca2+ interaction in contractility of vascular smooth muscle: Mg2+ versus organic calcium channel blockers on myogenic tone and agonist-induced responsiveness of blood vessels. Can J Physiol Pharmacol 1987 April;65(4):729-45

76. Quamme G A, de Rouffignac C. Epithelial magnesium transport and regulation by the kidney. Front Biosci 2000 Aug. 1;5:D694-711

77. M O Enkvist and K D McCarthy, Astroglial sap junction communication is increased by treatment with either glutamate or high K concentration. J Neurochem, 1994 February; vol. 62(2), 489-495.

79. Marilee K. Shelton and Ken D. McCarthy. Hippocampal Astrocytes Exhibit Ca-Elevating Muscarinic Cholinergic and Histaminergic Receptors in Situ. Journal of Neurochemistry, Vol. 74, No. 2, 2000 555-563.

80. Massry and Glassock's textbook of nephrology 4^{th},ed. By lippincott williams&wilkins. P 368

81. Massry and Glassock's textbook of nephrology 4^{th},ed. By lippincott williams&wilkins. P372 nephrology

82. Massry and Glassock's textbook of nephrology 4^{th},ed. By lippincott williams&wilkins. P373 nephrology

83. Massry and Glassock's textbook of nephrology 4^{th},ed. By lippincott williams&wilkins. P374-375 nephrology.

84. Massry and Glassock's textbook of nephrology 4^{th},ed. By lippincott williams&wilkins. P 378 nephrology.

85. Massry and Glassock's textbook of nephrology 4^{th},ed. By lippincott williams&wilkins. P349 nephrology.

86. Massry and Glassock's textbook of nephrology 4^{th},ed. By lippincott williams&wilkins. P351 nephrology.

87. Massry and Glassock's textbook of nephrology 4^{th},ed. By lippincott williams&wilkins. P352 nephrology.

88. GUYTON & HALL Textbook of Medical Physiology 10^{th},ED. P342 physiology

89. Ganong. Review of medical physiology. 20^{th, ED. P}38

90. P323 from genes to cells by stephen R. Bolsover 1997.

91. Bernheim L, Bader C R. Human myoblast differentiation: Ca channels are activated by K channels. News physiol. Sci. 17:22-26, 2002.

92. In Kaiser-Permanente study of 181 cases and 181 controls, risk of prostate cancer decreased with increasing prediagnostic levels of circulating 1,25 (OH)2D.

93. Edward Giovannucci. Dietary influences of 1,25 (OH)2 vitamin D in relation to prostate cancer: A hypothesis. Cancer causes and control, 1998,9,pp.567-582.

94. Increased fruit consumption and decreased intake of calcium does reduce the risk of advanced prostate cancer.Suman Kapur. A medical Hypothesis: Phosphorus Balance and Prostate Cancer. Cancer Investigation, 18(7),664-669(2000).

95. Giovannucci E &et. Calcium and fructose intake in relation to risk of prostate cancer. Cancer Res 1998Feb. 1;58(3):442-7.

96. Barri Y M, Knochel J P. Hypercalcemia and Electrolyte disturbance in malignancy. Hematologyloncology clinics of north america vol.10 No. 10:775-790,Augustl 1996.

97. Sartori L, Insogna K L, Barrett P Q. Renal phosphate transport in humoral Hypercalcemia of malignancy. Am J Physiol 1988 December;255(6pt 2): f1078-84.

98. Milionis H J, Bourantas C L, Siamopoulos K C, Elisaf M S. Acid-base and electrolyte abnormalities in patients with acute leukemia. Am J Hematol 1999 December ;62(4):201-7.

99. Sahin G, Ertem U, Duru F, Birgen D, Yuksek N. High prevalence of chronic magnesium deficiency in T cell lymphoblastic leukemia and malignant lymphoma. Leuk Lymphoma 2000 November;39(5-6):555-62.

100. Kopanski Z, Piekoszewski W, Schlegel-Zawadzka M, Wojewoda T, Szuszko R. the importance of determination of magnesium concentration in the serum of patients with cancer metastases to the liver. Przegl Lek 2002;59(4-5):267-8.

101. McKee M D, Cecco S A, Niemela J A, Cormier J, Kim C J, Steinberg S M, Rehak N N, Elin R J, Rosenberg S A. Effects of interleukin 2 therapy on lymphocyte magnesium levels. J Lab Clin Med 2002 January;139(1):5-12.

102. Kumar A R, Kurup P A. Membrane Na+K+ ATPase inhibition related dyslipidemia and insulin resistance in neuropsychiatric disorders. Indian J Physiol Pharmacol 2001 July;45(3):296-304.

103. Peracchia C, et al. Calmodulin Directly Gates Junction Channels. J.Biol. Chem., Vol.275, Issue 34, 26220-26224, Aug. 25, 2000.

104. Gopalakrishna R, Gundimeda U.Antioxidant regulation of protein kinase C in cancer prevention. J Nutr 2002 December;132(12):3819S-23S

105. Uzzo R G, Leavis P, Hatch W, Gabai V L, Dulin N, Zvartau N, Kolenko V M. Zinc Inhibits Nuclear Factor-kappaB Activation and Sensitizes Prostate Cancer Cells to Cytotoxic Agents. Clin Cancer Res 2002 November;8(11):3579-83

106. Lin Y, Kikuchi S, Obata Y, Yagyu K.Serum Copper/Zinc Superoxide Dismutase (Cu/Zn SOD) and Gastric Cancer Risk: a Case-Control Study. Jpn J Cancer Res 2002 October;93(10):1071-5

107. Kuo H W, Chen S F, Wu C C, Chen D R, Lee J H. Serum and tissue trace elements in patients with breast cancer in Taiwan. Biol Trace Elem Res 2002 October;89(1):1-11

108. Park K S, Ahn Y, Kim J A, Yun M S, Seong B L, Choi K Y. Extracellular zinc stimulates ERK-dependent activation of p21(Cip/WAF1) and inhibits proliferation of colorectal cancer cells. Br J Pharmacol 2002 November;137(5):597-607

109. Fang Y Z, Yang S, Wu G. Free radicals, antioxidants, and nutrition. Nutrition 2002 October;18(10):872-9

110. Syed Nazeer, MD Torsade de Pointes eMedicine Journal, Apr. 1, 2002, Volume 3, Number 4

111. Hartness M E, Lewis A, Searle G J, O'Kelly I, Peers C, Kemp P J.Combined antisense and pharmacological approaches implicate hTASK as an airway O(2) sensing K(+) channel. J Biol Chem 2001 Jul. 13;276(28):26499-508

112. O'Kelly I, Lewis A, Peers C, Kemp P J. O(2) sensing by airway chemoreceptor-derived cells. Protein kinase c activation reveals functional evidence for involvement of NADPH oxidase. J Biol Chem 2000 Mar. 17;275(11):7684-92

113. Lewis A, Peers C, Ashford M L, Kemp P J.Hypoxia inhibits human recombinant large conductance, Ca(2+)-activated K(+) (maxi-K) channels by a mechanism which is membrane delimited and Ca(2+) sensitive.J Physiol 2002 May 1;540(Pt 3):771-80

114. Wojtczak L, Teplova V V, Bogucka K, Czyz A, Makowska A, Wieckowski M R, Duszynski J, Evtodienko Effect of glucose and deoxyglucose on the redistribution of calcium in ehrlich ascites tumour and Zajdela hepotoma cells and its consequences for mitochondrial energetics. Further arguments for the role of Ca(2+) in the mechanism of the crabtree effect. Eur J Biochem 1999 July;263(2):495-501

115. Malhi H, Irani A N, Rajvanshi P, Suodicani S O, Spray D C, McDonald T V, Gupta S. KATP channels regulate mitogenically induced proliferation in primary rat hepatocytes and human liver cell lines. Implications for liver growth control and potential therapeutic torgeting.J Biol Chem 2000 Aug. 25;275(34):26050-7

116. Wojtczak L, Nikitino E R, Czyz A, Skulskii I A. Cuprous ions activate Slibenclamide-sensitive potassium channel in liver mitochondria. Biochem Biophys Res Commun 1996 Jun. 14;223(2):468-73

117. Bogucko K, Teplova W, Wojtczak L, Evtodienko W V, Wojtczaka L [corrected to Wojtczak L. Inhibition by Ca2+ of the hydrolysis and the synthesis of ATP in Ehrlich ascites tumour mitochondria: relation to the Crabtree effect. Biochim Biophys Acta 1995 Mar. 14; 1228(2-3):261-6

118. Evtodienko Yu V, Teplova V V, Duszynski J, Bogucka K, Wojtczak L.The role of cytoplasmic [Ca2+] in glucose-induced inhibition of respiration and oxidative phosphorylation in Ehrlich ascites tumour cells: a novel mechanism of the Crabtree effect. Cell Calcium 1994 June;15(6):439-46

119. Babenko*, L. Aguilar-Bryan,# J. Bryan*. A VIEW OF SUR/K ir6.X, Katp CHANNELS. Annu. Rev. Physiol. 1998.60:667-687.

120. Pharmacol.Ther.Vol.69, No.3, pp. 199-217, 1996

121. Ouadid-Ahidouch H, Le Bourhis X, Roudbaraki M, Toillon R A, Delcourt P, Prevarskaya N. Changes in the K+ current-density of MCF-7 cells during progression through the cell cycle: possible involvement of a h-ether.a-gogo K+ channel. Receptors Channels 2001;7(5):345-56

122. Hao X M, Fu Y, Jin H, Liu T F. Calcium-activated potassium current in single Novikoff cell. Methods Find Exp Clin Pharmacol 2001 March;23(2):55-9

123. Morklund L, Henriksson R, Grankvist K. Cisplotin-induced apoptosis of mesothelioma cells is affected by potassium ion flux modulator amphotericin B and bumetanide. Int J Cancer 2001 Aug. 15;93(4):577-83

124. Sheader E A, Brown P D, Best L. Swelling-induced changes in cytosolic [Ca2++] in insulin-secreting cells: a role in regulatory volume decrease? Mol Cell Endocrinol 2001 Jul. 5;181(1-2):179-87

125. Rybalchenko V, Prevarskaya N, Van Coppenolle F, Legrand G, Lemonnier L, Le Bourhis X, Skryma R. Verapamil inhibits proliferation of LNCaP human prostate cancer cells influencing K+ channel gating. Mol Pharmacol 2001 June;59(6):1376-87

126. Kim J A, Kang Y Y, Lee Y S. Activation of Na(+), K(+), Cl(−)-cotransport mediates intracellular Ca(2+) increase and apoptosis induced by Pinacidil in HepG2 human hepatoblastoma cells. Biochem Biophys Res Commun 2001 Feb. 23;281(2):511-9

127. Rouzaire-Dubois B, Milandri J B, Bostel S, Dubois J M. Control of cell proliferation by cell volume alterations in rat C6 glioma cells. Pflugers Arch 2000 October;440(6):881-8

128. Rosenboum C, Kamleiter M, Grafe P, Kluwe L, Mautner V, Muller H W, Hanemann C O. Enhanced proliferation and potassium conductance of Schwann cells isolated from NF2 schwannomas can be reduced by quinidine. Neurobiol Dis 2001 February;8(1):181

129. Hougaard C, Niemeyer M I, Hoffmann E K, Sepulveda F V. K+ currents activated by leukotriene D4 or osmotic swelling in Ehrlich ascites tumour cells. Pflugers Arch 2000 June;440(2):283-94

130. Fraser S P, Grimes J A, Djamgoz M B. Effects of voltage-gated ion channel modulators on rat prostatic cancer cell proliferation: comparison of strongly and weakly metastatic cell lines. Prostate 2000 Jun. 15;44(1):61-76

131. Choi B Y, Kim H Y, Lee K H, Cho Y H, Kong G. Clofilium, a potassium channel blocker, induces apoptosis of human promyelocytic leukemia (HL-60) cells via Bcl-2-insensitive activation of caspose-3. Cancer Lett 1999 Dec. 1;147(1-2):85-93

132. Kim J A, Kang Y S, Jung M W, Kang G H, Lee S H, Lee Y S. Ca2+ influx mediates apoptosis induced by 4-aminopyridine, a K+ channel blocker, in HepG2 human hepatoblastoma cells. Pharmacology 2000 February;60(2):74-81

133. Schwab A, Reinhardt J, Schneider S W, Gassner B, Schuricht B. K(+) channel-dependent migration of fibroblasts and human melanoma cells. Cell Physiol Biochem 1999;9(3):126-32

134. cycle in MCF-7 human breast cancer cells. J Membr Biol 1999 Sep. 1;171(1):35-46

135. Skryma R, Van Coppenolle F, Dufy-Barbe L, Dufy B, Prevarskaya N. Characterization of Ca(2+)-inhibited potassium channels in the LNCaP human prostate cancer cell line. Receptors Channels 1999;6(4):241-53

136. Soroceanu L, Manning T J Jr, Sontheimer H. Modulation of glioma cell migration and invasion using Cl(−) and K(+) ion channel blockers. J Neurosci 1999 Jul. 15;19(14):5942-54

137. Yao X, Kwan H Y. Activity of voltage-gated K+ channels is associated with cell proliferation and Ca2+ influx in carcinoma cells of colon cancer. Life Sci 1999;65(1):55-62

138. Costa R S, Assreuy J. Nitric oxide inhibits irreversibly P815 cell proliferation: involvement of potassium channels. Cell Prolif 2002 December;35(6):321-32

139. Pillozzi S, Brizzi M F, Balzi M, Crociani O, Cherubini A, Guasti L, Bartolozzi B, Becchetti A, Wanke E, Bernabei P A, Olivotto M, Pegoraro L, Arcangeli A. HERG potassium channels are constitutively expressed in primary human acute myeloid leukemias and regulate cell proliferation of normal and leukemic hemopoietic progenitors. Leukemia 2002 September;16(9):1791-8

140. Abdul M, Hoosein N. Expression and activity of potassium ion channels in human prostate cancer. Cancer Lett 2002 Dec. 1;186(1):99-105

141. O'Reilly N, Xia Z, Fiander H, Tauskela J, Small D L. Disparity between ionic mediators of volume regulation and apoptosis in N1E 115 mouse neuroblastoma cells. Brain Res. 2002 Jul. 12;943(2):245-56.

142. Piekarska A E, Webster L, Saltis J, McPherson G A. KATP channel blocking actions of quaternary ions play no role in their antiproliferative action on mouse leukaemia and rat vascular smooth muscle cells in vitro. Clin Exp Pharmacol Physiol 1998 December;25(12):992-8

143. Fieber L A. Ionic currents in normal and neurofibromatosis type 1-affected human Schwann cells: induction of tumor cell K current in normal Schwann cells by cyclic AMP. J Neurosd Res 1998 Nov. 15;54(4):495-506

144. Friederich P, Urban B W. The inhibition of human neuronal K+ currents by general anesthetic agents is altered by extracellular K. Brain Res Mol Brain Res 1998 Oct 1;60(2):301-4

145. O'Kelly I, Peers C, Kemp P J. O2-sensitive K+ channels in neuroepithelial body-derived small cell carcinoma cells of the human lung. Am J Physiol 1998 October;275(4 Pt 1):L709-16

146. Kawase T, Burns D M. Calcitonin gene-related peptide stimulates potassium efflux through adenosine triphosphate-sensitive potassium channels and produces membrane hyperpolarization in osteoblastic UMR106 cells. Endocrinology 1998 August;139(8):3492-502

147. Rouzaire-Dubois B, Dubois J M. K+ channel block-induced mammalian neuroblastoma cell swelling: a possible mechanism to influence proliferation. J Physiol 1998 Jul. 1;510 (Pt 1):93-102

148. Riquelme G, Sepulveda F V, Jorgensen F, Pedersen S, Hoffmann E K. Swelling-activated potassium currents of Ehrlich ascites tumour cells. Biochim Biophys Acta 1998 Apr. 22;1371(1):101-6

149. Tosetti P, Taglietti V, Toselli M. Functional changes in potassium conductances of the human neuroblastoma cell line SH-SY5Y durins in vitro differentiation. J Neurophysiol 1998 February;79(2):648-58

150. Wondergem R, Cregon M, Strickler L, Miller R, Suttles J. Membrane potassium channels and human bladder tumor cells: II. Growth properties. J Membr Biol 1998 Feb. 1;161(3):257-62

151. Yellowley C E, Hancox J C, Skerry T M, Levi A J. Whole-cell membrane currents from human osteoblost-like cells. Calcif Tissue Int 1998 February;62(2):122-32

152. Uppmann B J, Yang R, Barnett D W, Misler S. Pharmacology of volume regulation following hypotonicity-induced cell swelling in clonal N1E115 neuroblastoma cells. Brain Res 1995 Jul. 17;686(1):29-36

153. Brismar T, Anderson S, Collins V P. Mechanism of high K+ and Tl+ uptake in cultured human glioma cells. Cell Mol Neurobiol 1995 June;15(3):351-60

154. Wan S X, Greer M A. Blocking K+ channels with TEA induces plasmalemma depolarization, increased [Ca2+]i, and ACTH secretion in AtT-20 cells. Mol Cell Endocrinol 1995 Mar;109(1):11-8

155. Woodfork K A, Wonderlin W F, Peterson V A, Strobl J S. Inhibition of ATP-sensitive potassium channels causes reversible cell-cycle arrest of human breast cancer cells in tissue culture. J Cell Physiol 1995 February;162(2):163-71

156. Lohr J W, Yohe L A. Mechanisms of hypoosmotic volume regulation in glioma cells. Brain Res 1994 Dec. 26;667(2):263-8

157. Toral J, Hu W, Yi L, Barrett J E, Sokol P T, Ziai M R. Use of cultured human neuroblastoma cells in rapid discovery of the voltage-gated potassium-channel blockers. J Pharm Pharmacol 1994 September;46(9):731-4

158. Basovappa S, Romano-Silva M A, Mangel A W, Laro D, Campbell I, Brammer M. Inhibition of K+ channel activity by 4-AP stimulates N-type Ca2+ channels in CHP-100 cells. Neuroreport 1994 Jun. 2;5(10):1256-8

159. Weiger T, Hermann A. Polyamines block Ca(2+)-activated K+ channels in pituitary tumor cells (GH3). J Membr Biol 1994 June;140(2):133-42

160. Draheim H, Repp H, Malettke N, Dreyer F. Potassium single-channel properties in normal and Rous sarcoma virus-transformed chicken embryo fibroblasts. Pflugers Arch 1994 May;427(1-2):17-23

161. Verheugen J A, Oortgiesen M, Vijverberg H P. Veratridine blocks voltage-gated potassium current in human T lymphocytes and in mouse neuroblastoma cells. J Membr Biol 1994 February;137(3):205-14

162. HuanS X Y, Morielli A D, Peralta E G. Tyrosine kinase-dependent suppression of a potassium channel by the G protein-coupled m1 muscarinic acetylcholine receptor. Cell 1993 Dec. 17;75(6):1145-56

163. Nillus B, Schwarz G, Droogmans G. Control of intracellular calcium by membrane potential in human melanoma cells. Am J Physiol 1993 December;265(6 Pt 1):C1501-10

164. Hirsh J K, Quandt F N. Aminopyridine block of potassium channels in mouse neuroblastoma cells. J Pharmacol Exp Ther 1993 November;267(2):604-11

165. Lee Y S, Sayeed M M, Wurster R D. Inhibition of cell growth and intracellular Ca2+ mobilization in human brain tumor cells by Ca2+ channel antagonists. Mol Chem Neuropathot 1994 June;22(2):81-95

166. Lee Y S, Sayeed M M, Wurster R D. Inhibition of cell growth by K+ channel modulators is due to interference with agonist-induced Ca2+ release. Cell Signal 1993 November;5(6):803-9

167. Koong A C, Giaccia A J, Hahn G M, Saad A H. Activation of potassium channels by hypoxia and reoxygenation in the human lung adenocarcinoma cell line A549. J Cell Physiol 1993 August;156(2):341-7

168. Pancrazio J J, Tabbara I A, Kim Y I. Voltage-activated K+ conductance and cell proliferation in small-cell lung cancer. Anticancer Res 1993 July-August;13(4):1231-4

169. Medrano S, Gruenstein E. Mechanisms of regulatory volume decrease in UC-11MG human astrocytoma cells. Am J Physiol 1993 May;264(5 Pt 1):C1201-9

170. Erulkar S D, Ludmir J, Ger B, Nori R D. Expression of different potassium channels in cells isolated from human myometrium and leiomyomas. Am J Obstet Gynecol 1993 May;168(5):1628-39.

171. Lu L, Yang T, Markakis D, Gugino W B, Craig R W. Alterations in a voltage-gated K+ current during the differentiation of ML-1 human myeloblastic leukemia cells. J Membr Biol 1993 Mar;132(3):267-74

172. White R E, Lee A B, Shcherbatko A D, Lincoln T M, Schonbrunn A, Armstrong D L. Potassium channel stimulation by natriuretic peptides through cGMP-dependent dephosphorylation. Nature 1993 Jan. 21;361(6409):263-6

173. Wang Y F, Jia H, Walker A M, Cukierman S. K-current mediation of prolactin-induced proliferation of malignant (Nb2) lymphocytes. J Cell Physiol 1992 July;152(1):185-9

174. I to Y, Yokoyama S, Higashida H. Potassium channels cloned from neuroblastoma cells display slowly Inactivating outward currents in Xenopus oocytes. Proc R Soc Land B Biol Sci 1992 May 22;248(1322):95-101

175. Reeve H L, Vaughan P F, Peers C. Glibenclamide inhibits a voltage-gated K+ current in the human neuroblastoma cell line SH-SY5Y. Neurosci Lett 1992 Jan. 20; 135(1):37-40

176. Grissmer S, Lewis R S, Cahalan M D. Ca(2+)-activated K+ channels in human leukemic T cells. J Gen Physiol 1992 January;99(1):63-84

177. Maehlen J, Wallen P, Love A, Norrby E, Kristensson K. Paramyxovirus infections alter certain functional properties in cultured sensory neurons. Brain Res 1991 Feb. 1;540(1-2):123-30

178. Wu H, Franklin C C, Kim H D, Turner J T. Regulation of calcium-activated potassium efflux by neurotensin and other agents in HT-29 cells. Am J Physiol 1991 January;260(1 Pt 1):C35-42

179. Enomoto K, Furuya K, Yamagishi S, Oka T, Maeno T. Release of arachidonic acid via Ca2+ increase stimulated by pyrophosphonucleotides and bradykinin in mammary tumour cells. Cell Biochem Funct 1995 December;13(4):279-86

180. Enomoto K, Furuya K, Yamagishi S, Oka T, Maeno T. The increase in the intracellular Ca2+ concentration induced by mechanical stimulation is propagated via release of pyrophosphorylated nucleotides in mammary epithelial cells. Pflugers Arch 1994 July;427(5-6):533-42

181. Enomoto K, Furuya K, Yamagishi S, Maeno T. Mechanically induced electrical and intracellular calcium responses in normal and cancerous mammary cells. Cell Caldum 1992 August;13(8):501-11

182. Enomoto K, Furuya K, Maeno T, Edwards C, Oka T. Oscillating activity of a calcium-activated K+ channel in normal and cancerous mammary cells in culture. J Membr Biol 1991 January;119(2):133-9

183. Nilius B, Bohm T, Wohirab W. Properties of a potassium-selective ion channel in human melanoma cells. Pflugers Arch 1990 Nov;417(3):269-77

184. Robbins J, Sim J A. A transient outward current in NG108-15 neuroblastoma x glioma hybrid cells. Pflugers Arch 1990 April;416(1-2):1307

185. Leonard R J, Karschin A, Jayashree-Aiyar S, Davidson N, Tanouye M A, Thomas L, Thomas G, Lester H A. Expression of Drosophila Shaker potassium channels in mommalion cells infected with recombinant vaccinia virus. Proc Natl Acad Sci USA 1989 October;86(19):7629-33

186. Kunzelmann K, Pavenstadt H., Beck C, Unal O, Emmrich P, Arndt H J, Greger R.Lehrstuhl II Institut fur Physiologie, Albert-Ludwigs-Universitat, Freiburg, Federal Republic of Germany. Characterization of potassium channels in respiratory cells. I. General properties. Pflugers Arch 1989 July;414(3):291-6

187. Kunzelmann K, Pavenstadt H. Greger R. Characterization of potassium channels in respiratory cells. II. Inhibitors and regulation. Pflugers Arch 1989 July;414(3):297-303

188. Brismar T, Anderson S, Collins V P. Mechanism of high K+ and Tl+ uptake in cultured human glioma cells. Cell Mol Neurobiol 1995 June;15(3):351-60

189. Brismar T, Collins V P. Potassium and sodium channels In human malignant Stioma cells. Brain Res 1989 Feb. 20;480(1-2):259-67

190. Brismar T, Collins V P. Inward rectifying potassium channels in human malignant glioma cells. Brain Res 1989 Feb. 20;480(1-2):249-58

191. Quandt F N. Three kinetically distinct potassium channels in mouse neuroblastoma cells. J Physiol 1988 January;395:401-18

192. Lopez-Barneo J, Hoshi T, Heinemann S H, Aldrich R W. Effects of external cations and mutations in the pore region on C-type inactivation of Shaker potassium channels. Receptors Channels 1993; 1(1):61-71

193. Hoshi T, Garber S S, Aldrich R W. Effect of forskolin on voltage-gated K+ channels is independent of adenylate cyclase activation. Science 1988 Jun. 17;240(4859):1652-5

194. Hoshi T, Aldrich R W. Voltage-dependent K+ currents and underlying single K+ channels in pheochromocytoma cells. J Gen Physiol 1988 January;91(1):73-106

195. Korn S J, Weight F F. Patch-clamp study of the caldum-dependent chloride current In AtT-20 pituitary cells. J Neurophysiol 1987 December;58(6):1431-51

196. Ashford M L, Sturgess N C, Trout N J, Gardner N J, Hales C N. Adenosine-5′-triphosphate-sensitive ion channels in neonatal rat cultured central neurones. Pflugers Arch 1988 August;412(3):297-304

197. Sturgess N C, Hales C N, Ashford M L. Calcium and ATP regulate the activity of a non-selective cation channel in a rat insulinoma cell line. Pflugers Arch 1987 August;409(6):607-15

198. Sturgess N C, Ashford M L, Cook D L, Hales C N. The sulphonylurea receptor may be an ATP-sensitive potassium channel. Lancet 1985 Aug. 31;2(8453):474-5

199. Liepins A, Younghusband H B. A possible role for K+ channels in tumor cell injury. Membrane vesicle shedding and nuclear DNA fragmentation. Exp Cell Res 1987 April;169(2):385-94

200. Light D B, Van Eenenaam D P, Sorenson R L, Levitt D G. Potassium-selective ion channels in a transformed insulin-secreting cell line. J Membr Biol 1987;95(1):63-72

201. Bolsover S R. Two components of voltage-dependent calcium influx in mouse neuroblastoma cells. Measurement with arsenazo III. J Gen Physiol 1986 August;88(2):149-65

202. Higashida H, Streaty R A, Klee W, Nirenberg M. Bradykinin-activated transmembrane signals are coupled via No or Ni to production of inositol 1,4,5-trisphosphate, a second messenger in NG108-15 neuroblastoma-glioma hybrid cells. Proc Natl Acad Sci USA 1986 February;83(4):942-6

203. Supino R, Gibelli N, Galatulas I, Zunino F. Influence of calcium and calcium-modulating agents on differentiation of murine erythroleukaemia cells. Cell Biol Int Rep 1985 December;9(12):1059-68

204. Dunne M J, Findlay I, Petersen O H. Effects of pyridine nucleotides on the gating of ATP-sensitive potassium channels in insulin-secreting cells. J Membr Biol 1988 June;102(3):205-16

205. Findlay I, Dunne M J. ATP maintains ATP-inhibited K+ channels in an operational state. Pflugers Arch 1986 August;407(2):238-40

206. Dunne M J, Findlay I, Petersen O H, Wollheim C B. ATP-sensitive K+ channels in an insulin-secreting cell line are inhibited by D-glyceraldehyde and activated by membrane permeabilization. J Membr Biol 1986;93(3):271-9

207. Findlay I, Dunne M J, Ullrich S, Wollheim C B, Petersen O H. Quinine inhibits Ca2+-independent K+ channels whereas tetraethylammonium inhibits Ca2+-activated K+ channels in insulin-secreting cells. FEBS Lett 1985 Jun. 3;185(1):4-8

208. Findlay I, Dunne A U, Petersen O H. High-conductance K+ channel in pancreatic islet cells can be activated and inactivated by internal calcium. J Membr Biol 1985;83(1-2):169-75

209. Findlay I, Dunne M J, Petersen O H. ATP-sensitive inward rectifier and voltage- and calcium-activated K+ channels in cultured pancreatic Islet cells. J Membr Biol 1985;88(2):165-72

210. Vyklicky L Jr, Michl J, Vlachova V, Vyklicky L, Vyskocil F. Ionic currents in neuroblastoma clone E-7 cells. Neurosci Lett 1985 Apr. 9;55(2):197-201

### BRIEF SUMMARY OF THE INVENSION

Decrease energy inside a cell will turn it into cancerous cell. Treating this cell will phosphate and glucose Will help replenish energy sources inside that cell. The cell will need Magnesium and Potassium to restore what was lost that it can function normal again. Verapamil will help to counteract the effects of increase intracellular Ca on the cell.

DETAILED DESCRIPTION OF THE INVENTION
THE PROTOCOL OF A STUDY TO CONFIRM THE CLAIM.

```
  1. Patient Name
  2. Address
  3. DOB
  4. Sex Race
  5. Cancer Type Duration Stage
  6. Treatment taken before for cancer
  7. Medicines taking at the present time.
  8. Lab done for Ca, Mg, Ph, K, Na, BUN, Cr, Bicarb, EKG, Hepatic
function.
  9. Urine K, PH, Mg, Ca.
10. Height, weight, Bp, pulse, Temp.
```

Exclusion Criteria

1. Patients with hyperphosphatemia, Hyperkalemia and patients with hypermagneseimia.

2. Patients with renal failure, heart block, addison disease, myocardial damage, sever hepatitis, crush syndrome and adrenocortical insufficiency, hypocalcemia.

Inclusion Criteria for First Stage.

1. Patients with any cancer already diagnosed.

2. Other therapy has been tried before and failed or could not continued for other reasons.

3. Patients who are willing to participate and signed release form.

4. Age 18 or above.

Questions to Know the Underlying Causes.

1. Antacids.

2. Diarrhea: how often it happened, how long it last, how many bowel movements in a day.

3. Diabetus Melitus.

4. Hospitalization/ICU/TPN/Bone marrow transplant.

5. Eating Disorders.

6. Alcoholism: how often the drinking, how much.

7. Exposure to Heavy metals

8. Drugs include loop diuretics, cisplatinum, panidronate, Acetazolamide and glucocorticoids.

9. Extensive burns.

10. Dental or swallowing difficulties.

11. Primary intestinal disorders such as Crohn disease or celiac sprue.

12. Forced saline diuresis, extracellular volume expansion or administration of bicarbonate.

13. Vitamin D Deficiency: poor oral intake, lack of sun exposure

14. Hyperventilation with acute respiratory alkalosis: salicylate overdose, panic attacks, and sepsis.

15. Radiation injury to the bowel, bowel resection, small bowel bypass.

16. Laxative abuse, IBD,

17. Diuretics: thiazide, loop diuretics.

18. Cisplatin, pentamidine,

19. Floride poisoning

20. Primary aldosteronism, hypoparathyroidism, hyperthyroidism.

21. Excessive lactation, for how long.

22. Pregnant women esp. with preterm labor.

23. Occult diuretic use/polyuria: how many times per day/how much each.

24. Vomiting how often, how much

25. Secondary hyperaldosteronism due to volume depletion, CHF, Cirrhosis or vomiting

26. Occult laxative use, bulimia

27. Renal artery stenosis

28. Licorice ingestion

29. Some penicillins, Amphotericin B, Gentamicin.

30. High-dose beta agonists therapy (COPD)

31. Non-potassium sparing diuretics

32. AIDS

33. Hyperthrodism, Immobilization, thiazides, Vit A intoxication, Renalfailure which leads to high bone turnover.

34. Vit D intoxication

35. Granulomatous disorders eg. Sarcoidosis

36. Hyperparathyrodism

37. Subcutanous fat necrosis.

THE GOAL OF THE STUDY

If the cellular abnormalities came from low energy, reversing that will correct the problem. When the cells return to normal, no more proliferation or metastasis would be expected. The cells should start to respond to normal stimuli around. It might be possible that tumor might then decrease in size or disappear as in those situations that tumors resolve spontaneously without reason.

DURATION:

No comparable study was done to show how long it will take for the cells to return to normal. From personal experience, patients with phosphate depletion respond with marked symptomatic improvement within 2 weeks. Lab. Results takes longer to return to normal. If the study last 3 months, it might be enough to see not only symptomatic improvement but also physical improvements.

STUDY DESIGN

1. I would like to start open study on a small number of patients. Then if works, we might do another one on a larger scale on all age groups.

2. The patients will have blood and urine tests before the enrollment. They will also be assessed for their cancer stage and Cancer complications.

3. The patients will receive neutrophosphate 250 mg QID, Mg 500 mg QD, K gluconate 50 mEq/day, Verapamil (Verelan PM) 200 mg/day.

4. Patients need to keep a daily symptom diary for cancer symptoms and for adverse reactions. Any patients experiencing significant adverse reactions will be withdrawn from the study.

5. Follow up with these patients on weekly bases with blood work and imaging studies if indicated depending on the type of cancer. Daily blood work or shorter periods might be needed in some situations. Clinical assessment will be needed on a weekly base.

6. Outcome measures will include

1. Rate of resolution of symptoms and the tumor.

2. Incidence of adverse reactions to medications.

3. Incidence of clinical relapse during the three months following treatment.

BUDGET

The cost will include the blood and urine tests,

Clinical assessment visits.

Imaging studies, bone marrow or other tests as indicated.

Since these testing are usually done for these cancer patients. These tests might be covered under their insurance.

Lab.

Blood: Serum calcium, magnesium, phosphate, potassium, Na, Serum albumin, serum bicarbonate, serum glucose, BUN and creatinine, Creatine kinase.

Serum phosphate and calcium should be monitored every 6 hours to weekly depending on the serum levels to ensure maintenance of normal calcium levels and to prevent overcorrection of phosphate in sever phosphate depletion. urinalysis.

Urinalysis for amino acids (proteinuria) and glucose.

A 24-hour urine collection for phosphate, magnesium, calcium, potassium and sodium.

plain bone films, ECG and cardiac monitor

MEDICATIONS

For less severe hypophosphatemia (1-2 mg/dL), PO phosphate salt preparations can be used.

Adult Dose Initial dose:0.1 mmol/kg of K 2 PO 4 or Na 2 PO 4 q6h IV (32 mmol/d)

Aggressive IV replacement:0.2-0.3 mmol/kg of K 2 PO 4 or Na 2 PO 4 over 6 h

For oral replacement, Neutra-Phos packets contain 250 mg as capsule, liquid, or packet tid/qid is generally adequate.

Pediatric Dose 0.25-0.5 mmol/kg PO over 4-6 h; repeat if symptomatic hypophosphatemia persists

Potassium gluconate

adults iv 10-40 mEq/2-3 hrs. 50-100 mEq/day on 1, 2, 3 doses.

Pediatrics 0.5-1 mEq/kg not more than 30-40 mEq/dose.not to exceed 0.3-0.5/kg/hr. 2-4 mEq/kg/d in divided doses.

Mg gluconate, 500 mg (27 mg elemental) po qd

pediatrics 3-6 mg elemental/kg/d po 3/d,4/d not to exceed 400 mg/24 hr.

Fructose or glucose as tolerated.

Verapamil (Verelan PM) 200-400 mg as tolerated.

What we claim as our invension is:

1. THE CAUSE OF CANCER IS DECREASE THE ENERGY INSIDE A CELL WHICH CHANGE IT FROM NORMAL CELL TO A CANCEROUS CELL. INCREASING THE ENERGY INSIDE THAT CELL WILL RESTORE THE CELL TO ITS NORMAL CONDITION.
